(19) United States
(12) Patent Application Publication
(10) Pub. No.: US 2022/0151887 A1
(43) Pub. Date: May 19, 2022

(54) HYDROGEN INHALATION COSMETIC METHOD, AND HIGH-CONCENTRATION HYDROGEN INHALATION DEVICE USED IN HYDROGEN INHALATION COSMETIC METHOD

(71) Applicant: AQUA BANK CO.,LTD., Osaka-shi Osaka (JP)

(72) Inventor: Takashi TAKEHARA, Osaka-shi Osaka (JP)

(21) Appl. No.: 17/440,517

(22) PCT Filed: Mar. 19, 2020
(86) PCT No.: PCT/JP2020/012519

§ 371 Date: Sep. 17, 2021

(30) Foreign Application Priority Data
Mar. 19, 2019 (JP) 2019-051912

(51) Int. Cl. A61K 8/19; A61Q 19/08; A61Q 19/02; A61M 15/02; A61M 15/08

(52) U.S. Cl. CPC A61K 8/19; A61Q 19/08; A61Q 19/02; A61M 2205/8206; A61M 15/08; A61K 2800/805; A61M 15/02

(57) ABSTRACT

A method of beautification using hydrogen inhalation capable of promoting improvement of skin condition by regular intake of a high-concentration hydrogen-containing gas air into a body by oral or nasal inhalation under spontaneous breathing, and a high-concentration hydrogen inhalation device appropriate for use in the method of beautification using hydrogen inhalation are provided. According to the method of beautification using hydrogen inhalation, regular oral or nasal inhalation of a high-concentration hydrogen-containing gas generated from hydrogen generating means can non-therapeutically promote improvement of skin condition. In addition, the high-concentration hydrogen inhalation device for use in the method of beautification using hydrogen inhalation includes: hydrogen generating means that generates and releases hydrogen; and hydrogen transporting means that guides the high-concentration hydrogen-containing gas to the nose and mouth of a human body, the high-concentration hydrogen-containing gas being a mixture of hydrogen released from the hydrogen generating means and environmental air.

### TECHNICAL FIELD

The present invention relates to a method of beautification using hydrogen inhalation in which regular intake of a high-concentration hydrogen-containing gas air into a body by oral or nasal inhalation under spontaneous breathing can promote improvement of skin condition, and to a high-concentration hydrogen inhalation device appropriate for use in the method of beautification using hydrogen inhalation.

### BACKGROUND ART

Recently, effectiveness of hydrogen has attracted attention in human clinical tests, and various studies have been actively conducted in medical applications. Methods of administering hydrogen to a human body include intravenous dosage, oral administration of an aqueous solution, and gas inhalation (nasal and oral inhalation), and there are wide variety of methods for taking hydrogen into the body.

However, no clear medical trial case has been conventionally provided for gas inhalation in methods of administering hydrogen. Recently, hydrogen inhalation at medical institutions has attracted attention, and there have been provided trial cases on what kind of vital reaction is actually caused by inhalation intake of hydrogen and patent documents as the result (Non-Patent Literature 1 etc.).

Under these circumstances, the inventor, who is a provider of devices that allow inhalation of hydrogen on a daily basis, has focused on vital reactions caused by “regular inhalation” of hydrogen and has conducted various trial cases. The inventor has provided, for example, demonstration results in which it is recognized that brain stress is reduced and there are remarkable influences on physical functions (Patent Literature 1), and demonstration results in which it is recognized that living functions are improved and cognitive functions are prevented and/or improved in those who are suspected of having mild cognitive impairment (MCI).

On the other hand, hydrogen intake has conventionally attracted attention in the beautification industry as well, and a method of oral intake of hydrogen-containing drinking water (what is called hydrogen solution) is known. However, there are few trial cases such that improvement of a specific site is clearly recognized by oral intake, and this has been thought to lead to misunderstandings about the intake of hydrogen into the body and factors that hinder the promotion of development. Actually, in the case of oral intake of what is called hydrogen solution, it has been difficult to maintain the hydrogen content in hydrogen-containing drinking water and to control the amount of intake into the body so that it has been difficult to conduct a demonstration test that could be a trial case.

Under these circumstances, the inventor has demonstrated and developed a hydrogen inhalation device that can give a sufficient intake amount of hydrogen into the body and that can provide a variety of trial cases for skin conditions and can repeatedly cause a good result. Here, the inventor has determined that the target of the vital reaction by inhalation administration of hydrogen is the skin condition, which is easy to control the intake amount and time, and is a site attracting attention in the beautification field.

### CITATION LIST

### Patent Literature

Patent Literature 1: International Publication No. WO 2018/151107 A1

### Non Patent Literature

Hayashi K et al.: Inhalation of hydrogen gas reduces infact size in the rat model of myocardial Ischemia-reperfusion injury. Biochem.Biophsy.Res.Commun, 373: 30-35, 2008.

### SUMMARY OF INVENTION

### Technical Problem

Therefore, an object of the present invention is to provide a method of beautification using hydrogen inhalation in which it is possible to promote improvement of skin condition by intake of a high-concentration hydrogen-containing gas air into the body by regular oral or nasal inhalation under spontaneous breathing for a predetermined time or longer each time and continuously for a predetermined period of time, and to provide a high-concentration hydrogen inhalation device appropriately configured for use in the method of beautification using hydrogen inhalation.

### Solution to Problem

In order to solve the aforementioned problems, a first aspect of the present invention provides a method of beautification using hydrogen inhalation for non-therapeutically promoting improvement of skin condition, the method including regularly performing oral or nasal inhalation of a high-concentration hydrogen-containing gas generated from hydrogen generating means.

As in the method of beautification using hydrogen inhalation of the first aspect of the present invention, regular intake of the high-concentration hydrogen-containing gas air by oral or nasal inhalation into the body under spontaneous breathing can promote improvement of skin condition. Conventionally, improvement of living function and cognitive function when hydrogen is orally or nasally inhaled has been verified. This time, regular inhalation of the hydrogen-containing gas, such as inhalation several times every day, and for several minutes each time, is found to promote improvement of skin condition, as is to be described below. Here, the predetermined time each time, which is a single time for hydrogen inhalation, means about an inhalation time in which a transitory pupil contraction reaction is generally observed.

In addition, in the method of beautification using hydrogen inhalation, reduction in redness and decrease in a number of red spots and brown spots can typically promote improvement of skin condition. Specifically, of the skin conditions, red spots and brown spots were clearly improved by inhalation of the high-concentration hydrogen-containing gas (see FIGS. 9 and 10A-10B).

In addition, in the method of beautification using hydrogen inhalation, increase in a return rate and viscoelasticity of the skin can promote improvement of the skin condition. As described above, in addition to red spots and brown spots, increase in skin return rate (Uf=R2) and skin viscoelasticity (R7) were demonstrated as a remarkable improvement example by inhalation of the high-concentration hydrogen-containing gas (continuous use). The R2 is Ua/Uf, which is the ratio of Ua to Uf, where Uf is the maximum height of the skin drawn into the probe of the measuring instrument under negative pressure, and Ua is the height returned from the skin height at maximum extension after the negative pressure is released; and The viscoelasticity R7 of the skin is an immediate (instantaneous) contraction rate with respect to the stretched length of the skin in inhaling a high-concentration hydrogen-containing gas, which is equal to the elastic modulus during contraction (R7=Ur/Uf); and Ur/Uf is a return elasticity rate obtained by analysis of the displacement of the skin that has deformed in a constant suction pressure and time.

In addition, in the method of beautification using hydrogen inhalation, oral inhalation or nasal inhalation of the hydrogen-containing gas is preferably performed for 5 minutes or more each time, at intervals of about 15 minutes or more, 5 times or more every day, and continuously for about 2 weeks or more.

For example, inhaling hydrogen 5 times or more every day for about 5 minutes each time, and continuously performing this for about 2 weeks or more can surely promote improvement of the aforementioned skin conditions. In the subsequent crossover test, which is to be described below, the skin condition of the subjects who stopped inhalation of high-concentration hydrogen gas for two weeks and the other subjects who newly performed inhalation of high-concentration hydrogen gas for two weeks were reversed. This clearly indicates that inhalation of high-concentration hydrogen gas 5 times every day, continuously for 2 weeks, for at least about 5 minutes each time, in which a transitory pupil contraction reaction is observed, promotes improvement of skin condition. It was also found that a certain effect was obtained by inhaling air containing high-concentration hydrogen gas at least about 15 minutes apart, for example, “upon getting up, after breakfast, after lunch, after dinner, before going to bed”.

In addition, the hydrogen-containing gas to be orally or nasally inhaled preferably contains 1 to 2% of hydrogen.

Next, a second aspect of the present invention is a high-concentration hydrogen inhalation device for use in the aforementioned method of beautification using hydrogen inhalation, the device including:

hydrogen generating means that generates and releases hydrogen;

hydrogen transporting means that guides the high-concentration hydrogen-containing gas to the nose and mouth of a human body, the high-concentration hydrogen-containing gas being a mixture of hydrogen released from the hydrogen generating means and environmental air; and

an attachment for use for oral inhalation or nasal inhalation of a high-concentration hydrogen-containing gas from the hydrogen transporting means.

The high-concentration hydrogen inhalation device of the second aspect of the present invention is a device for orally or nasally inhaling air containing high-concentration hydrogen gas as a dedicated product for use for improving the skin condition. As described above in the first aspect of the present invention, regular intake of the high-concentration hydrogen-containing air into the body by oral or nasal inhalation under spontaneous breathing can promote improvement of skin condition, and the high-concentration hydrogen inhalation device, as a dedicated device that could appropriately achieve the improvement, adopts various characteristic configurations.

First, the high-concentration hydrogen inhalation device has hydrogen generating means for generating hydrogen and mixing the generated hydrogen with environmental air, and can generate a hydrogen-containing gas having an appropriate concentration for inhalation. The generated hydrogen-containing gas having an appropriate concentration is transported to the nose and mouth by the hydrogen transporting means, and can be introduced into the nose and mouth from an attachment that can be connected to the nose and mouth. Therefore, it is possible to promote improvement of the skin condition as in the demonstration example to be described below.

A specific example of the high-concentration hydrogen inhalation device is a portable hydrogen inhalation assembly including the hydrogen generating means, the hydrogen transporting means, and the attachment;

wherein: the hydrogen generating means has an electrolysis tank that generates hydrogen by energizing a positive electrode and a negative electrode that are separated from each other in water to be electrolyzed and by electrolyzing the water to be electrolyzed; and

the hydrogen transporting means and the attachment are formed into an integrally coupled mixing portion that guide a high-concentration hydrogen-containing gas to the nose and mouth by negative pressure generated by oral inhaling or nasal inhaling, the high-concentration hydrogen-containing gas being a mixture of hydrogen generated in the electrolysis tank and environmental air.

This high-concentration hydrogen inhalation device is portable and adopts an electrolytic type that reliably allows inhalation of a desired amount of hydrogen by energization as a hydrogen generation method. In order to improve the skin condition, it is necessary to regularly inhale the high-concentration hydrogen-containing air, for example, to perform inhalation 5 times or more every day for a predetermined time each time, at intervals of about 15 minutes or more, and continuously for about 2 weeks or more, etc. Therefore, it is essential for the device to have portability so that the device can be easily used anywhere such as at home, and the high-concentration hydrogen inhalation device having this configuration is preferable for such requirements. In the demonstration example to be described below, which actually exhibited an advantageous effect, the device having the same configuration is used. Furthermore, the high-concentration hydrogen inhalation device is advantageously provided with the mixing portion in which the hydrogen transport means that mixes hydrogen and environmental air and the attachment that introduces the mixed air into the nose and mouth are integrally configured to adjust the concentration of hydrogen so that the concentration can be adjusted to an appropriate level by the negative pressure of normal inhalation.

Furthermore, this high-concentration hydrogen inhalation device is a device including

a body cover member that can be orally or nasally inhaled while being grasped with one hand, the body cover member including a battery, a control substrate that controls power supply from the battery, a pair of positive/negative electrodes in which the positive electrode and negative electrode are energized or not energized by the control substrate,

wherein: the electrolysis tank is attached to the body cover member, has the pair of positive/negative electrodes inserted inside, and is a transparent body or translucent body capable of storing water;

the mixing portion is detachably attached to an upper portion of the electrolysis tank and is integrally configured to have a nozzle portion and a channel, the nozzle portion guiding the hydrogen-containing gas air into the nose and mouth, the channel fluidically connecting the electrolysis tank to the nozzle portion and taking in environmental air; and

the control substrate preferably controls power supply and stop from the battery to the positive/negative electrodes by operating one set of operation means disposed on a side portion of the body cover member.

The high-concentration hydrogen inhalation device adopts a configuration in which at least operations related to hydrogen generation/stop can be easily operated in one item of operation means by a hand grasping the device at the time of hydrogen inhalation so that any user can easily use the device. Furthermore, in the case of electrolytic hydrogen generation, bubbles of hydrogen and oxygen are generated in the electrolysis tank, and a transparent or translucent electrolysis tank would allow a user who has an impaired cognitive function and ability for continuing one task to understand the state of bubble generation and to easily understand to be inhaling good gas. This is also advantageous in that caregivers easily understand.

### Advantageous Effects of Invention

According to the method of beautification using hydrogen inhalation of the first aspect of the present invention, regular oral or nasal inhalation of the high-concentration hydrogen-containing gas air under spontaneous breathing, for example, inhaling several times every day for several minutes each time, can promote improvement of skin conditions as in the method of beautification using hydrogen inhalation.

The high-concentration hydrogen inhalation device of the second aspect of the present invention is an appropriate device for use in the method of beautification using hydrogen inhalation of the first aspect of the present invention, and it is capable of generating a hydrogen-containing gas at an appropriate concentration for inhalation to promote improvement of skin condition, of transporting it to the nasal opening and introducing it into the nose and mouth.

### BRIEF DESCRIPTION OF DRAWINGS

FIG. 1 is a block diagram schematically illustrating an embodiment of the high-concentration hydrogen inhalation device of the present invention.

FIGS. 2A-2D are front view, left and right side views, and a plan view illustrating an embodiment of the high-concentration hydrogen inhalation device of the present invention.

FIG. 3 is a schematic diagram illustrating a state of electrolysis in an electrolysis tank of the high-concentration hydrogen inhalation device of the present invention.

FIG. 4 schematically illustrates a specific configuration of an electrode.

FIGS. 5A-5C are an external photographic views of an example of the embodiment of the high-concentration hydrogen inhalation device of the present invention.

FIG. 6 is a diagram illustrating a change in pupil contraction rate with hydrogen inhalation time.

FIG. 7 is a diagram illustrating changes in skin temperature at a peripheral site by measuring skin temperature.

FIG. 8 is a diagram illustrating changes in skin viscoelasticity.

FIG. 9 is a diagram illustrating changes in skin spots and brown spots.

FIGS. 10A-10B are diagrams illustrating an effect on redness of skin.

### DESCRIPTION OF EMBODIMENTS

First, description is to be made below on an example of a method of beautification using hydrogen inhalation that non-therapeutically promotes improvement of skin condition according to a first aspect of the present invention (hereinafter, also simply referred to as “the method of beautification using hydrogen inhalation”), and the demonstration results thereof.

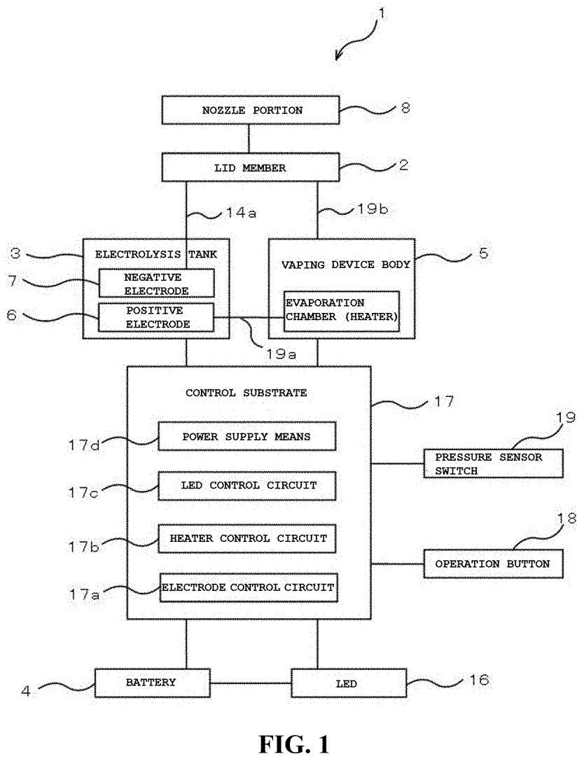

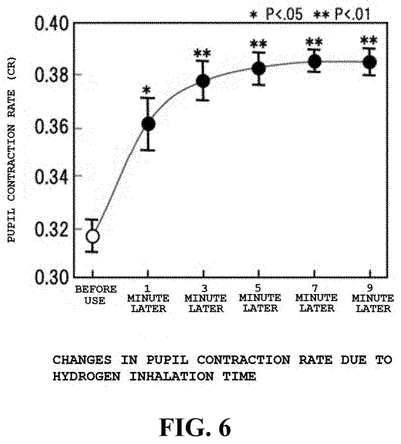

In the verification test using the method of beautification using hydrogen inhalation, subjects orally inhaled hydrogen generated by hydrogen generating device for improving health (high-concentration hydrogen inhalation device 1 to be described below (“Kencos 3” manufactured by Aqua Bank Co., Ltd. (see FIGS. 1 to 5A-5C))). Specifically, the subjects inhaled the hydrogen generated by the high-concentration hydrogen inhalation device 1 under spontaneous breathing. Note that the high-concentration hydrogen inhalation device generates 8 cc of hydrogen gas per minute by electrolyzing water by the electrolysis to be described below (at the same time, the device generates 4 cc of oxygen gas as well), so that the device generates 12 cc of oxygen/hydrogen gas mixture in one minute. Since an adult normally inhales approximately five liters of air per minute in spontaneous breathing, it is theoretically calculated that a maximum of 0.24% (hydrogen: 0.18%, oxygen: 0.06%) of the gas mixture is contained in the inhaled gas, assuming that all the generated gas mixture is inhaled. The hydrogen gas to be inhaled was adjusted for each subject each time, and the high-concentration hydrogen inhalation device once used was not reused. In addition, in order to calculate the optimum hydrogen inhalation amount, the pupil reaction to light were measured after inhalation for 1 minute, 3 minutes, 5 minutes, 7 minutes and 9 minutes. As a result, the pupil contraction rate in the pupil reaction to light due to hydrogen inhalation increased significantly in a time-dependent manner (FIG. 6). It was clarified that the pupil contraction rate significantly increased with hydrogen inhalation for up to 3 minutes, and the effect stabilized with hydrogen inhalation for 7 minutes and more. Therefore, in the subsequent tests, it was determined to examine physiological reactions each for hydrogen inhalation for five minutes.

Note that the increases in concentrations of the gas mixture of hydrogen and oxygen generated from the high-concentration hydrogen inhalation device 1 are 0.18% for hydrogen and 0.06% for oxygen as described above, while the concentrations in the atmosphere are 0.5×10⁻⁴% (=0.5 ppm) for hydrogen and about 21% for oxygen. Since the increase in oxygen concentration in the gas mixture is very small, the verification results can be considered to be substantially due to the increase in hydrogen concentration.

In the single time evaluation test of hydrogen inhalation, the subjects were 17 healthy women aged 25 to 40 years. The selection criteria were good health and no smoking. The subjects were allowed to sit in a sitting position for 30 minutes after entering a test room set at a room temperature of 24° C. and a relative humidity of 50% to acclimatize to the experimental environment. After acclimation, physiological measurements were conducted. As the physiological measurements, skin temperature measurements were performed. Each measurement was conducted under spontaneous breathing (representing a control) and after a hydrogen inhalation for 5 minutes (representing a preparation), and the measurement results were analyzed. Then, by comparing and examining the measured values, the physiological effect of inhaling hydrogen gas was verified.

<<Evaluation of Pupil Contraction Rate in Pupil Reaction to Light by Measuring Pupil Reaction to Light (Single Time Evaluation Test (FIG. 6))>>

In a method of measuring the pupil contraction rate in the pupil reaction to light (FIG. 6) by measuring the pupil reaction to light in which the appropriate hydrogen gas inhalation time is evaluated as described above, a subject was first adapted to darkness for 5 minutes in a state in which she wore a goggle-like electronic pupillometer (Iriscorder Dual, manufactured by Hamamatsu Photonics K.K.) on the face for shading. After confirming that the pupil dilatation was stable, light stimulation was given for 0.1 seconds. A transitory pupil contraction reaction was observed due to pupil reflection to light. The pupil contraction rate (CR), which is the rate of pupil contraction, was determined by the following expression:

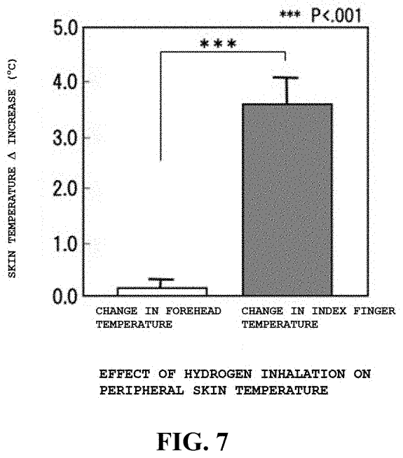

(D1−D2)/D1

where D1 is the initial state pupil diameter before light stimulation, and D2 is the pupil diameter after light stimulation for either the control (air inhalation for 5 minutes) or the sample (hydrogen inhalation for 5 minutes), and D1−D2 is the amount of change. Note that the pupil rapidly dilates after it contracts, and the pupil expanding speed (vd) at that time reflects the state of sympathetic nervous activity. The predominance of sympathetic nervous activity and parasympathetic nervous activity in autonomous nervous activity was determined by analysis of changes in pupil diameter. FIG. 6 shows that the pupil contraction rate CR significantly increased after hydrogen inhalation, especially after 5 minutes or more of inhalation, as compared with that before inhalation (p<0.001). On the other hand, the pupil expanding speed (vd) was almost unchanged (not shown).

<<Evaluation of Sympathetic Nervous Activity by Measuring Skin Temperature ((Single Time Evaluation Test (FIG. 7))>>

Since many of the peripheral blood vessel networks are controlled by sympathetic nervous activity, the predominance or inferiority of sympathetic nervous activity can be evaluated by measuring changes in blood flow in the peripheral site or changes in skin temperature in the peripheral site. Therefore, in this verification, the change in skin temperature was used as the index. In other words, changes in the skin temperatures (at the center of the forehead and the first segment pad of the index finger) of the peripheral site were measured by a temperature sensor (manufactured by Gram Corporation). The increased skin temperature in the test product is determined by the following expression:

ΔT2=(F2−F1)−(H2−H1)

where H1 is the temperature of the forehead center part of the control, F1 is the temperature of the finger pad center part of the control, H2 is the temperature of the forehead center part of the sample, and F2 is the temperature of the finger pad center part of the sample. For example, if a significant increase in skin temperature is observed by hydrogen inhalation, it is presumed that sympathetic nervous activity becomes inferior by hydrogen inhalation. As a result of the test, as shown in FIG. 7, the skin temperature of the forehead was hardly found to change by hydrogen inhalation, but the skin temperature of the index finger was found to significantly increase by an average of 3.6° C. (p<0.001). There were subjects who had an increase of as much as 6° C. or more in the skin temperature of the index finger due to hydrogen inhalation.

Note that, in the measurement data analysis in the physiological evaluation, the difference between the control and the blank and the difference between the test product and the blank were compared using the t-test. The significance level of the t-test was set to less than 5%.

In the evaluation test on continuous use of hydrogen inhalation, the subjects were 22 healthy women aged 25 to 39 years living in and around Tokyo. The conditions for selecting the subjects were determined to be that they were in good health and did not smoke, that their skin properties deteriorated due to stress and fatigue so that, for example, they were aware of the less firmness and sagging of the skin and feel the dullness of the skin, and that they were in a mild stress state that corresponded to a stress level of 6 to 10 in 30 items of the brief stress checklist. During the test period, each subject was required to perform the same skin care method as she does on a daily basis, and not to add or change cosmetics or supplements.

In this verification test, the effect of improving skin properties was confirmed by using the high-concentration hydrogen inhalation device 1 and continuously using the hydrogen gas inhalation. The test period was determined to be a total of 4 weeks in which each subject used the high-concentration hydrogen inhalation device 1 continuously for 2 weeks and stopped using it for 2 weeks, and a comparative evaluation was conducted between the hydrogen nonuse period and the hydrogen use period. In other words, the subjects were divided into two groups, Group A and Group B each having 11 subjects. In Group A, the first 2 weeks was set to the hydrogen use period and the next 2 weeks the hydrogen nonuse period, and conversely, in Group B, the first 2 weeks was set to the hydrogen nonuse period and the next 2 weeks the hydrogen use period. Skin physiological measurements were conducted at the start of the test (zero week), the second and fourth weeks (actually, the subjects also answered questionnaires of the 30 items of the stress checklist and the multifaceted emotion scale for verification of influences on stress, which is a factor of increased skin metabolism, as is omitted here). The measurement items include: the stratum corneum water content; transepidermal water loss; skin viscoelasticity; skin color; facial image analysis by photographing with a digital imaging device (VSIA); and skin blood flow measurement with a non-contact blood flow meter. In the second and fourth weeks of the test, the aforementioned psychological questionnaire was answered and the skin physiological measurement was conducted, and the psychological and physiological effects of hydrogen inhalation were verified by comparing and examining each measured value.

Here, in the continuous use test of hydrogen inhalation, the high-concentration hydrogen inhalation device 1 was used as in the single time test described above. The subjects executed the inhalation for five minutes each time in the morning and evening and approximately 1 hour after each meal, as in the single time test, with the goal of using five times a day.

The skin physiological measurements include: the stratum corneum water content; transepidermal water loss; skin viscoelasticity; skin color; facial image analysis by photographing with a digital imaging device (VSIA); and skin blood flow measurement with a non-contact blood flow meter. These skin physiological measurements were performed at the zero week, the second week, and the fourth week, and the skin physiological effect of hydrogen inhalation was verified by comparing and examining each measured value. In the measurements listed below, the cheek site, which was the measurement site, was measured after 15 minutes of acclimation in a constant temperature and humidity chamber (temperature 20±2° C., humidity 50±10%) after the face washing. What was measured in each measurement are described below.

<<Measurement of Stratum Corneum Water Content and Transepidermal Water Loss (TEWL) (Evaluation Test on Continuous Use)>>

The degree of skin moisturization was determined by measuring the stratum corneum water content. The stratum corneum water content at the measurement site was measured using SKICON-200EX (manufactured by Yayoi Co., Ltd.). The measurement was performed 5 times on the cheek, and the average value of 3 measurements excluding the highest value and the lowest value was taken as the measured value. The measurements were performed at zero, second and fourth weeks. In addition, the degree of skin barrier ability was determined by measuring the transepidermal water loss. The TEWL at the measurement site was measured using a cyclone moisture transpiration meter AS-CT1 (manufactured by Asahi Biomed Co., Ltd.). The measurement was performed 5 times on the cheek, and the average value of 3 measurements excluding the highest value and the lowest value was taken as the measured value. The measurements were performed at zero, second and fourth weeks.

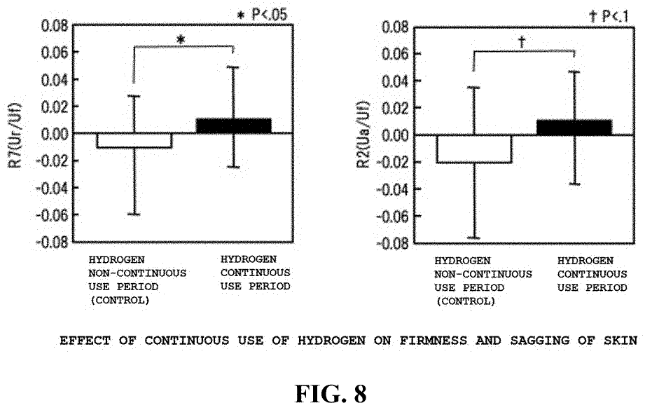

As a result of the test, an increase in skin water content was observed during the hydrogen continuous use period (difference average value; +34.9 μS), while almost no increase in water content was observed (difference average value; +6.5 μS) during the hydrogen non-continuous use period, but there was no significant difference, as is not illustrated. From this result, it was presumed that the subjects' skin water content was originally high and the skin was sufficiently moisturized so that no significant change was observed. In addition, a decrease in transepidermal water loss was observed during the hydrogen continuous use period (difference average value; +34.9), while an increase in water content was hardly observed during the hydrogen non-continuous use period (difference average value; +6.5), but there was no significant difference. From this result, it was presumed that the originally low transepidermal water loss of the subject sufficiently maintained the barrier function of the skin so that no change was observed.

<<Measurement of Skin Viscoelasticity (Evaluation Test on Continuous Use (FIG. 8))>>

The degree of firmness and sagging of the skin was determined by measuring the viscoelasticity of the skin. The skin viscoelasticity of the measurement site is measured by the Cutometer MPA580 (C+K electronic GmbH.) Since the dry state of the stratum corneum affects the measured value, a small amount of a commercially available cream preparation was applied to the measurement site about 15 minutes before the measurement to eliminate the influence of the dryness and wetness of the stratum corneum. The measurement was performed 5 times on the cheek, and the average value of 3 measurements excluding the highest and lowest values was determined to be the measured value. The measurements were performed at zero, second and fourth weeks.

R2=Ua/Uf, R6=Uv/Ue, and R7=Ur/Uf were analyzed as parameters of skin viscoelasticity. Uf represents the maximum height of the skin drawn into the probe when negative pressure was applied, and Ua represents the height returned from the skin height at maximum extension after the negative pressure was released. Ua/Uf represents the return rate of the skin. Ue (immediate distension) is an increase in skin height that rises linearly when negative pressure is applied, and Uv (delayed distension) is a non-linear increase in skin height that follows Ue. Ue mainly reflects the elasticity of the skin, Uv mainly reflects the viscosity of the skin, and Uv/Ue is said to represent the degree of viscosity that contributes to changes in the skin. Ur/Uf is said to represent biological elasticity, where Ur represents immediate retraction. It is suggested that as the skin elasticity increases, the R2 value increases, the R6 value decreases, and the R7 value increases.

As shown in FIG. 8, as a result of the test, the difference average value of the instantaneous skin return rate R7 (Ur/Uf) value was 0.01 during the hydrogen continuous use period, whereas it was −0.01 during the hydrogen nonuse period. The p value was 0.028, which was statistically significant, and the effect size was 0.74, indicating a moderate effect. Furthermore, the difference average value of the skin return rate R2 value was 0.01 during the hydrogen use period, whereas it was −0.02 during the hydrogen nonuse period. The p value was 0.076, which was not statistically significant, but the effect size was 0.59, indicating a moderate effect, so that it was determined that there was a significant tendency due to hydrogen continuous use. The fact that continuous use of hydrogen inhalation increases the instantaneous resilience of the skin and the skin return rate suggests that the firmness of the skin is restored and the sagging thereof is improved.

<<Skin Color Measurement (Evaluation Test on Continuous Use)>>

The skin color of the measurement site was measured using a handy color difference meter NR555 (manufactured by Nippon Denshoku Industries Co., Ltd.). The measurement was performed 7 times on the cheek, and the average value of 5 measurements excluding the highest and lowest values was taken as the measured value. The measurements were performed at zero, second and fourth weeks.

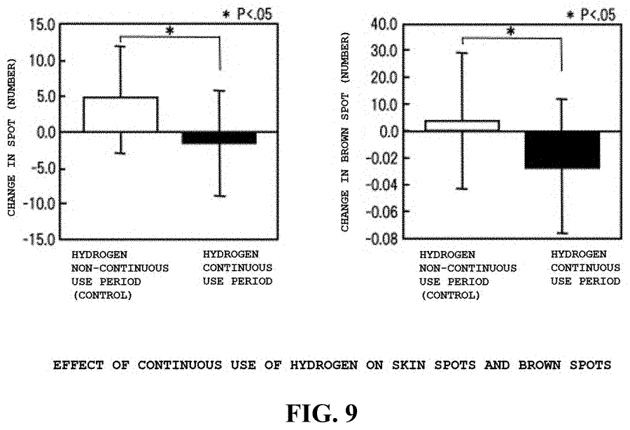

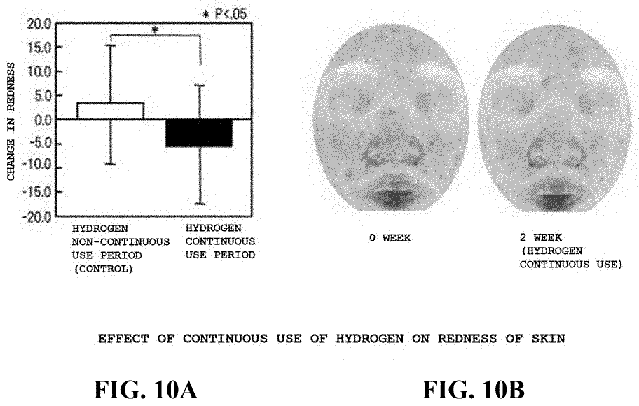

As a result of the test, the skin color of the measurement site was analyzed by dividing it into three factors (L*, a*, b*), as is not illustrated. In other words, the skin color was divided into black and white degree L*, redness degree a*, and yellowness degree b* for analysis. The result showed the difference average value of each factor was (ΔL*: +0.30, Δa*: −0.23, Δb*: −0.20) in the hydrogen continuous use period, whereas it was (ΔL*: −0.22, Δa*: −0.45, Δb*: −0.01) in the hydrogen non-continuous use period. This suggested that continuous use of hydrogen tended to improve the skin color by whitening the skin color, reducing redness, and reducing yellowness, but the difference was not significant.

<<Analysis of Skin Properties by VISIA (Registered Trademark)-Evolution (Evaluation Test on Continuous Use (FIG. 9 and FIGS. 10A-10B))>>

Skin images of the measurement site were photographed using VISIA (registered trademark) Evolution (Canfield Imaging Systems), and each skin property was analyzed. The software attached to the device was used to measure the scores of pores, spots, wrinkles, texture, redness, porphyrin, and brown spots. The measurements were performed at zero, second and fourth weeks.

As a result of the test, pores, spots, wrinkles, textures, redness, porphyrins, and brown spots were analyzed as skin properties by VISIA (registered trademark). Of these factors, the measured values of pores, wrinkles, and texture showed no difference between the hydrogen continuous use period and the hydrogen non-continuous use period (the results are not shown). It was observed that the number of porphyrins tended to decrease during the hydrogen continuous use period (difference average value; −103.05) as compared with the hydrogen non-continuous use period (difference average value; +190.68), but the difference was not significant.

In addition, in the analysis of skin spots, VISIA (registered trademark) detected color unevenness existing in the skin surface layer derived from excess melanin (from the VISIA Evolution operation manual). Therefore, the measured values of the analyzed spots and brown spots are considered to be indexes that indicate that the smaller the number of the spots, the more uniform the skin color. Then, when the spots were analyzed, the difference average value of the number of spots was −1.8 during the hydrogen continuous use period, whereas it was 4.7 during the hydrogen nonuse period. The p value was 0.0012, which was statistically significant, and the effect size was 0.86, indicating a great effect. In the analysis of brown spots, the difference average value in the number of brown spots was −13.7 during the hydrogen continuous use period, whereas it was 3.8 during the hydrogen nonuse period. The p value was 0.046, which was statistically significant, and the effect size was 0.68, indicating a moderate effect. These results are shown in FIG. 9.

On the other hand, in the analysis of the redness of the skin color, the difference average value of the redness was −5.1 during the hydrogen continuous use period, while it was 3.2 during the hydrogen non-continuous use period. The p value was 0.042, which was statistically significant, and the effect size was 0.65, indicating a moderate effect. The result is shown in FIG. 10A. In addition, a VISIA image is shown in FIG. 10B as an effective example of decrease in redness due to hydrogen continuous use for 2 weeks. The skin color measurement results and VISIA image analysis results suggest that continuous use of hydrogen reduces redness of facial skin and significantly decreases the numbers of spots and brown spots. Therefore, it is considered that the uniformization of the skin color contributed to less feeling of the dullness of the skin.

<<Measurement of Skin Blood Flow (Evaluation Test on Continuous Use)>>

The skin blood flow at the measurement site was measured using a two-dimensional laser blood flow imaging device (OMEGA ZONE OZ-2, manufactured by Omega Wave Co., Ltd.). The measurement site was the entire face. After a rest for 1 minute, the measurement was performed for 30 seconds, and the average value during that period was used as the measured value. The measurements were performed at zero, second and fourth weeks.

As a result of the test, when the change in skin blood flow of the entire face was analyzed using a two-dimensional laser blood flow imaging device, the difference average value in skin blood flow changes was −0.20 during the hydrogen continuous use period, whereas the difference average value in blood flow changes was −0.53 during the hydrogen nonuse period, as is not illustrated. From the difference, it was first considered that the continuous use of hydrogen prevented the decrease in skin blood flow, but the difference was finally found to be not significant.

Note that, in the data analysis, the difference value (A value) is calculated from the measured values before and after the hydrogen use period and the hydrogen nonuse period of the subjects in each group, and values, which are calculated by adding and averaging the difference values of the subjects in the hydrogen use period group and the hydrogen nonuse period group, were respectively determined to be the difference average value of the hydrogen use period group and the difference average value of the hydrogen nonuse period group.

The expressions for calculating the difference average values are as follows:

the difference average value of hydrogen use period group: AΔ (2W−0W)+BΔ (4W−2W);

the difference average value of hydrogen nonuse period group: AΔ (4W−2W)+BA (2W−0W)

The p-value was calculated as the probability to determine whether there was a statistically significant difference in the difference average values of the respective groups, and the effect size was calculated as an index representing the magnitude of the difference between the difference average values, so that the two groups are compared with each other.

<<Example of High-Concentration Hydrogen Inhalation Device>>

Next, a second aspect of the present invention is a hydrogen generating device recommended for performing methods of living body improvement that improves living function and/or cognitive function according to the first aspect of the present invention, and a typical embodiment of the second aspect of the present invention is to be described in detail below with reference to FIGS. 1-5A and 5B. However, it goes without saying that the high-concentration hydrogen inhalation device 100 is not limited to the device illustrated in FIGS. 1 to 5A-5B.

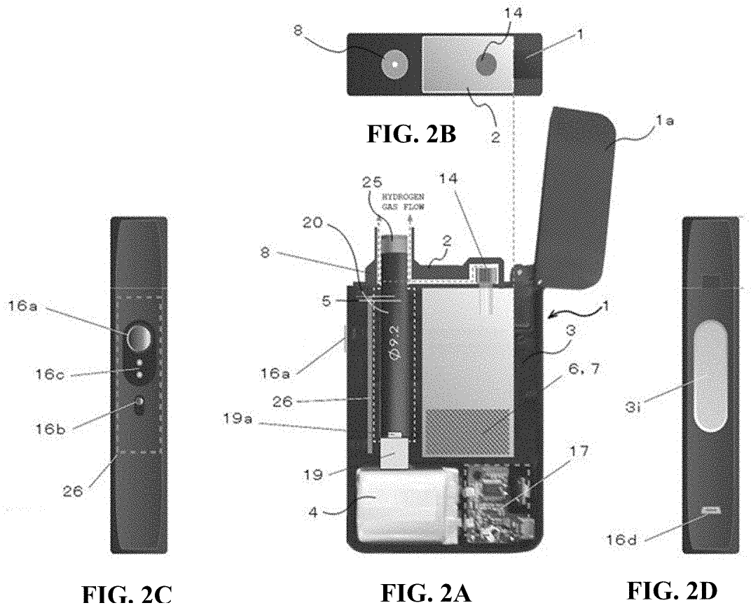

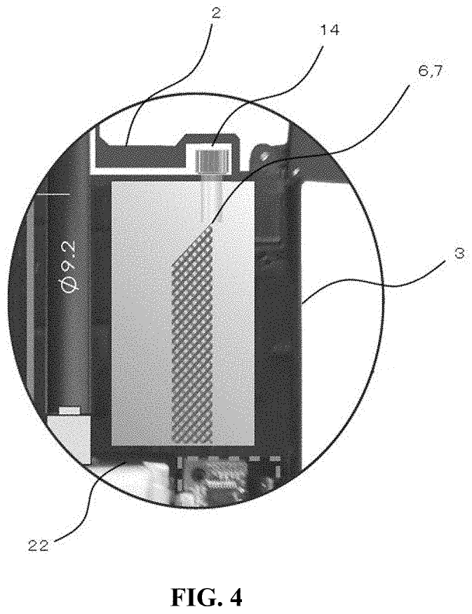

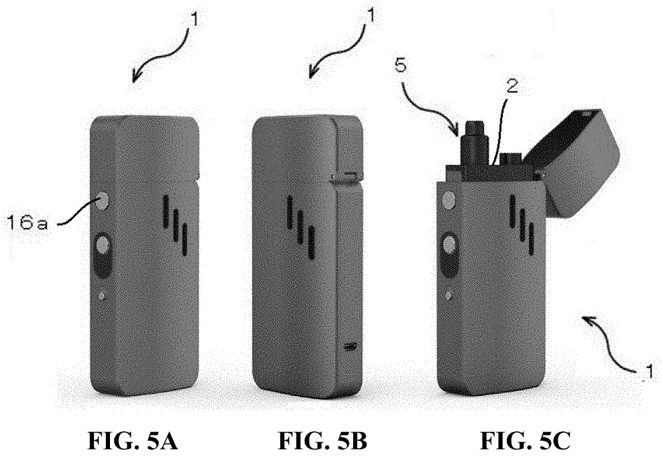

The embodiment of the high-concentration hydrogen inhalation device 1 is to be described below as an example. FIG. 1 illustrates a block diagram schematically illustrating the embodiment, FIGS. 2A-2D illustrate six views of a typical example of the embodiment of FIG. 1, and FIG. 4 illustrates a schematic diagram illustrating a state of electrolysis in an electrolysis tank of the high-concentration hydrogen inhalation device. FIGS. 5A-5C schematically illustrates a specific configuration of the electrode. Note that it is needless to say that the high-concentration hydrogen inhalation device 1 of the present invention is not limited to the device illustrated in the drawing, and also includes a device in which anything mentioned in the illustration and description is modified within the range of common sense.

As shown in FIG. 1, the high-concentration hydrogen inhalation device includes a battery 4, an LED 16, control means 17, an electrolysis tank 3, an aroma or supplement cartridge (hereinafter, also simply referred to as “cartridge”) 5, a mixing portion 2, and a nozzle portion 8. The battery 4 is of a charging type, and a pair of positive/negative electrodes 6 and 7 are disposed in the electrolysis tank 3. The positive/negative electrodes 6 and 7 are supplied with power from the battery 4 via the control means 33, and the LED 16 is connected to the battery 4. The control means 17 includes an electrode control circuit 17a, a heater control circuit 17b, an LED control circuit 17c, and power supply means (a power supply circuit) 17d.

A pressure sensor switch 19 is provided at the bottom portion of the receiving portion of the cartridge 5, and when the lower end of the cartridge presses the pressure sensor switch 19, the power of the battery 4 is supplied to the cartridge 5 by the power supply means 17d of the control substrate 17.

When the user operates the operation button 18, the electrode control circuit 17d accordingly controls energization/non-energization of a pair of electrodes 6 and 7 in the electrolysis tank 3, and the electric power supply means 17d varies the amount of power supplied from the battery 4 to supply power to the electrodes 6 and 7. When power is supplied to the pair of electrodes 6 and 7, the water stored in the electrolysis tank 10 is electrolyzed, oxygen is generated on the positive electrode 6 side, and hydrogen is generated on the negative electrode 7 side.

Hydrogen generated from the negative electrode 7 flows into the lid member 2 via the attachment 14 on the upper part of the electrolysis tank 3. The oxygen generated from the positive electrode 6 is vented.

In the cartridge 5, when the pressure sensor switch 19 is turned on, the heater inside the cartridge 5 is supplied with power from the battery 4 by the power supply means 17d, and the heater heats a cartridge, attached inside an internal vapor chamber (not shown), that has adsorbed an aromatic component or a supplement having a health and beauty promoting effect. When the heater heats the cartridge that has adsorbed a supplement (including a drug) or an aromatic component (hereinafter, also simply referred to as “supplement”), supplement-containing vapor is generated.

The supplement-containing vapor generated in the cartridge 5 is released into the mouth by inhaling the nozzle portion 8. At this time, due to the negative pressure generated by inhaling, hydrogen released from the attachment 4 flows in the lid member 2, and passes through the gap between the periphery of the upper part of the cartridge 5 exposed in the lid member 2 and the inner wall of the nozzle portion 8, mixes with the supplement-containing air, and is guided into the mouth. It is also conceivable to generate supplement-containing vapor by heating this.

FIGS. 2A-2D illustrate a specific configuration example of the high-concentration hydrogen inhalation device 1. FIG. 2A shows a front view of the high-concentration hydrogen inhalation device 1, FIG. 2B shows a top view, FIG. 2C shows a left side view, and FIG. 2D shows a right side view. FIG. 2A is a state in which the lid member 2 of the high-concentration hydrogen inhalation device 1 is removed, and there is a tubular cartridge receiving portion (hereinafter, also referred to as “receiving portion”) 20 extending downward from an opening on the upper right side with the lid member 2 removed (opened). The cartridge 5 is inserted into the receiving portion 20. The cartridge 5 is a substitute part for the body portion of a general-purpose tubular heating type vaping device.

When the upper part of the cartridge 5 is inhaled and negative pressure is generated, the cartridge 5 is turned on, and is supplied with power from the rechargeable battery in the battery 4 while the main power source, to be described below, is turned on, so that the heater heats the vapor chamber, releasing the supplement or aromatic component. Furthermore, in the cartridge 5, when the upper end is inhaled and a negative pressure is applied thereto, the LED 30b at the lower end of the battery 4 lights up at the same time while the power from the battery 4 is supplied.

Here, returning to FIG. 2A-2D, the high-concentration hydrogen inhalation device 1 is to be described. The cartridge 5 is inserted into the receiving portion 20 of the high-concentration hydrogen inhalation device 1. A pressure sensor switch 19 is disposed at the bottom portion of the receiving portion 20, and a convex screw 19a having the same shape as the attachment 30a is provided at the upper end of the pressure sensor switch 19 as an electric terminal. When the pressure sensor switch 19 is pressed, power from the rechargeable battery (lithium battery) 4 is supplied to the convex screw 19a, so that the supplement-containing vapor can be inhaled.

The right side portion (see FIG. 2C) of the high-concentration hydrogen inhalation device 1 is provided with a cartridge on/off switch 16c, an LED indicator 16b, and a main power source/hydrogen button 16a. The cartridge on/off switch 16c is an on/off switch of the pressure sensor switch 19, so that the power of the rechargeable battery 4 is supplied to the cartridge 5 when it is turned on and the attachment 5a at the lower end of the cartridge 5 is pressed with the convex screw 19 being connected thereto, but the power is not supplied from the rechargeable battery 4 when it is turned off even if the pressure sensor switch 19 is pressed. The main power source/hydrogen button 16a is a button-type power supply switch of the positive/negative electrodes 6 and 7 in the electrolysis tank 3, which is to be described below, and the main power source, and is used as both on/off of the main power source and on/off of power supply to the positive/negative electrodes 6 and 7 depending on the pressing manner/time.

In this example, when the main power source/hydrogen button 16a is held down for 3 seconds, the positive/negative electrodes 6 and 7 are energized for 5 minutes to generate hydrogen, and when it is pressed three times for two seconds, the main power source is turned off. The main power source is automatically turned off after 20 minutes even if the operation of turning it off is not performed. Furthermore, the main power source/hydrogen button 16a lights up while hydrogen is generated, and has a function of displaying the remaining amount of the rechargeable battery 4 according to the lighting color. In this example, when the remaining battery level is 20 to 80%, it lights up in blue, and when the remaining battery level is 80 to 100%, it lights up in white. In addition, the LED indicator 16b is provided with two LEDs each at upper and lower sides, the upper side LED lights up when power is supplied to the positive/negative electrodes 6 and 7 in the electrolysis tank 3, and the lower side LED lights up when the pressure sensor switch 19 is turned on and the cartridge 5 is energized. The lighting of the vaping device on/off switch 16c, the LED indicator 16b, and the main power source/hydrogen button 16 is controlled by the internal indicator substrate 26.

As described above, when the pressure sensor switch 19 is turned on, the power from the rechargeable battery 4 is also supplied to the pair of positive/negative electrodes 6 and 7 by the control substrate 17. As shown in FIG. 2C, the pair of positive/negative electrodes 6 and 7 may be disposed horizontally on the inner bottom portion of the electrolysis tank 5, or may be disposed vertically as shown in FIG. 1. The rechargeable battery 4 can receive power from the USB terminal 16d on the side portion of the high-concentration hydrogen inhalation device 1 and is charged (see FIG. 2D).

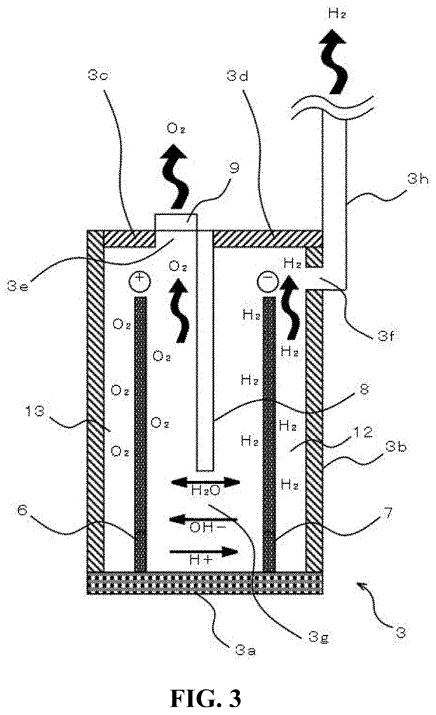

Next, with reference to FIG. 3, description is to be made on the configuration inside the electrolysis tank 3 and the state of electrolysis in the electrolysis tank 3 when the positive/negative electrodes 6 and 7 are energized. As shown in FIG. 3, the electrolysis tank 3 in which water is stored includes: a tubular member 3b that is hollow and longitudinally extends; a bottom member 3a that closes the bottom portion of the tubular member 3b; and the lid members 3c and 3d that close the upper portion of the tubular member 3a (3c and 3d may be integrally formed). When the positive/negative electrodes 6 and 7 are energized, oxygen (02) is generated in the vicinity of the positive electrode 6 and hydrogen (H₂) is generated in the vicinity of the negative electrode 7. Since the generated oxygen and hydrogen have a lighter specific gravity than water, they move upward and each move to a gap of 3g. Here, the electrolysis tank 3 is provided with a partition member 8 extending downward from the upper end thereof and dividing the electrolysis tank 3 into a hydrogen gas generating layer 12 on the negative electrode 7 side and an oxygen gas generating layer 13 on the positive electrode 6 side. The lower end of the partition member 8 is provided with a gap 3g from the upper surface of the bottom member 3a so as to fluidically connect the hydrogen gas generating layer 12 and the oxygen gas generating layer 13.

The partition member 8 prevents the mixing of oxygen and hydrogen in the electrolysis tank 3 during the upward movement of oxygen and hydrogen. On the other hand, in the lower part of the gap 3g provided in the lower part of the partition plate 8 which is not partitioned by the partition member 8, water (H₂O) can freely move, that is, ions (“OH⁻” and “H⁺”) can move, which is required for generation of oxygen and hydrogen. In this way, the partition member 50 achieves prevention of mixing of oxygen and hydrogen while performing electrolysis.

The lid member 3c closes the upper portion of the oxygen gas generating layer 13, but is provided with an opening 3e between a part of the lid member 3c or the lid member 3c and the partition member 8 or the tubular member 3b. The opening 3e is closed by the oxygen permeable membrane 9. Therefore, even if hydrogen leaks from the hydrogen gas generating layer 12 to the oxygen gas generating layer 13 due to the gap 3g or the like, the gas released to the outside by the oxygen permeable membrane 9 is limited to oxygen. The oxygen permeable membrane 9 may be disposed at the electrolytic solution injection port/hydrogen generation port 14 (to be described below) shown in FIGS. 2A-2D, but is preferably disposed in a hole dedicated to the electrolysis tank 3.

In addition, also in the hydrogen gas generating layer 12, the upper part of the hydrogen gas generating layer 12 is closed by the lid member 3d, but an opening 3f is provided in the upper part of the tubular member 3b on the hydrogen gas generating layer 12 side. The opening 3f is connected to the bypass channel 3h. Therefore, the hydrogen in the hydrogen gas generating layer 12 generated at the negative electrode 7 flows into the bypass channel 3h and flows upward.

Regarding the hydrogen channel from the opening 3f to the bypass channel 3h in FIG. 3, in the example of FIGS. 2A-2D, the electrolytic solution injection port/hydrogen generation port 14 corresponds to the opening 3f, and the gap between the upper portion of the electrolysis tank 3 and the lid member 2 corresponds to the bypass channel 3h. As described above, the electrolytic solution injection port/hydrogen generation port 14 has a function as an inlet for injecting the electrolytic solution or water into the electrolysis tank 5 and a function as an opening 3f for releasing hydrogen in the electrolysis tank 5 to the outside. The electrolytic solution injection port/hydrogen generation port 14 has a shape in which screws can be detachably fastened, and the electrolytic solution or water is injected through the opening 3f in a state where the screws are loosened and removed. In addition, leakage of the electrolytic solution and the like in the electrolysis tank 5 is prevented with the screws being fastened, but hydrogen is released from a permeable membrane for gas such as hydrogen (not shown) that closes a hole or the like separately provided in the electrolytic solution injection port/hydrogen generation port 14.

The released hydrogen flows in the lid member 2 in the left direction (cartridge 5 direction) as shown by the dotted line in FIGS. 2A-2D. The lid member 2 has a tubular nozzle portion 2a having an opening at the upper end and projecting at the left end, and is a removable integral member disposed on the upper part of the electrolysis tank 3, covering the upper end of the cartridge 5 and the electrolytic solution injection port/hydrogen generation port 14. When the lid member 2 is mounted on the upper part of the electrolysis tank 3, the nozzle portion 2a is in a state in which the upper end of the cartridge 5 is nested in the opening with a gap 26 around it. When the nozzle portion 2a is inhaled, hydrogen (high-concentration hydrogen-containing air) flowing from the electrolytic solution injection port/hydrogen generation port 14 rises in the gap 26 and is released to the outside (see the dotted line in FIGS. 2A-2D). This enables intake of high-concentration hydrogen. In addition, when power is supplied to the cartridge 5, this enables intake of a gas mixture of high-concentration hydrogen and air that contains a supplement having a health/beauty promoting effect and/or an aromatic component. The high-concentration hydrogen inhalation device 1 includes a cover 1a that can be opened and closed, and the example of FIGS. 2A-2D illustrate a state in which the cover 1a is opened. An opening (electrolytic solution check window) 3i for observing the liquid amount in the electrolysis tank 3 is provided on a side portion of the high-concentration hydrogen inhalation device 1 so that the amount of liquid in the electrolysis tank 3 can be visually recognized.

For reference, FIGS. 5A-5C show an external photographic view of an example of the embodiment of the high-concentration hydrogen inhalation device 1 of the present invention. FIG. 5A is a view of the high-concentration hydrogen inhalation device 1 viewed diagonally from the front left, FIG. 5B is a view of the high-concentration hydrogen inhalation device 1 viewed diagonally from the front right, and FIG. 5C shows a view of the high-concentration hydrogen inhalation device 1 in FIG. 5A viewed diagonally from the front left with the cover 1a being opened. Although there are some parts different from the example of FIGS. 2A-2D in terms of design, the main structure is substantially the same, and the reference numerals given to FIGS. 5A-5C are the same as those of the example of FIGS. 2A-2D.

The description and illustration are made above on the embodiments of the method of beautification using hydrogen inhalation according to the first aspect of the present invention and the high-concentration hydrogen inhalation device appropriate for performing the method of beautification using hydrogen inhalation according to the second aspect of the present invention, but the present invention is not limited to this, and those skilled in the art would understand that other modifications and improvements can be obtained without departing from the spirit and teachings described in the claims and the specification.

### INDUSTRIAL APPLICABILITY

According to the method of beautification using hydrogen inhalation that non-therapeutically promotes improvement of skin condition and the high-concentration hydrogen inhalation device appropriate for performing the method of beautification using hydrogen inhalation of the present invention, improvement of skin condition can be promoted by continuously orally inhaling or nasally inhaling the high-concentration hydrogen-containing gas air on a daily basis, and it is possible to provide the beautification industry with a new method for improving the skin condition, and to provide a device appropriate for use in the method, which can contribute to the expansion of the beautification market.

### REFERENCE SIGNS LIST

1 high-concentration hydrogen inhalation device
2 lid member
3 electrolysis tank
4 rechargeable battery
5 cartridge
6 positive electrode
7 negative electrode
8 nozzle portion
17 control means
18 operation button (operation means)
19 pressure sensor switch
19a convex screw
20 cartridge receiving portion
25 cartridge with supplement adsorbed

1. A method of beautification using hydrogen inhalation for non-therapeutically promoting improvement of skin condition, the method comprising regularly performing oral or nasal inhalation of a high-concentration hydrogen-containing gas generated by hydrogen generating means, the inhalation being performed continuously for a predetermined period of time, for a predetermined time or more each time.

2. The method of beautification using hydrogen inhalation according to claim 1, wherein reduction in redness and decrease in a number of red spots and brown spots promote improvement of skin condition.

3. The method of beautification using hydrogen inhalation according to claim 1, wherein increase in a return rate of skin and viscoelasticity of skin promote improvement of skin condition.

4. The method of beautification using hydrogen inhalation according to claim 1, wherein oral or nasal inhalation of the hydrogen-containing gas is performed at intervals of about 15 minutes or more, for five minutes or more each time, five times or more every day, and continuously for about 2 weeks or more.

5. The method of beautification using hydrogen inhalation according to a claim 1, wherein the hydrogen-containing gas to be orally or nasally inhaled contains 1 to 2% of hydrogen.

6. A high-concentration hydrogen inhalation device for use in the method of beautification using hydrogen inhalation according to claim 1, the device comprising:
hydrogen generating means that generates and releases hydrogen;
hydrogen transporting means that guides the high-concentration hydrogen-containing gas to the nose and mouth of a human body, the high-concentration hydrogen-containing gas being a mixture of hydrogen released from the hydrogen generating means and environmental air; and
an attachment for use for oral or nasal inhalation of a high-concentration hydrogen-containing gas from the hydrogen transporting means.

7. The high-concentration hydrogen inhalation device according to claim 6, the device comprising
a portable hydrogen inhalation assembly including the hydrogen generating means, the hydrogen transporting means, and the attachment,
wherein: the hydrogen generating means has an electrolysis tank that generates hydrogen by energizing a positive electrode and a negative electrode that are separated from each other in water to be electrolyzed and by electrolyzing the water to be electrolyzed; and
the hydrogen transporting means and the attachment are formed into an integrally coupled mixing portion that guides a high-concentration hydrogen-containing gas to the nose and mouth by negative pressure generated by oral or nasal inhaling, the high-concentration hydrogen-containing gas being a mixture of hydrogen generated in the electrolysis tank and environmental air.

8. The high-concentration hydrogen inhalation device according to claim 7, comprising
a body cover member that can be orally or nasally inhaled while being grasped with one hand, the body cover member including a battery, a control substrate that controls power supply from the battery, a pair of positive/negative electrodes in which the positive electrode and negative electrode are energized or not energized by the control substrate,
wherein: the electrolysis tank is attached to the body cover member, has the pair of positive/negative electrodes inserted inside, and is a transparent body or translucent body capable of storing water;
the mixing portion is detachably attached to an upper portion of the electrolysis tank and is integrally configured to have a nozzle portion and a channel, the nozzle portion guiding the hydrogen-containing gas air into the nose and mouth, the channel fluidically connecting the electrolysis tank to the nozzle portion and taking in environmental air; and
the control substrate controls power supply and stop from the battery to the positive/negative electrodes by operating one set of operation means disposed on a side portion of the body cover member.
